(19) United States
(12) Patent Application Publication
(10) Pub. No.: US 2005/0042214 A1
(43) Pub. Date: Feb. 24, 2005

(54) Discovery of the microorganism that causes the human autoimmune disease, primary biliary cirrhosis

(76) Inventors: M. Eric Gershwin, Davis, CA (US); Patrick S.C. Leung, Davis, CA (US)

Correspondence Address: GRAY CARY WARE & FREIDENRICH LLP, 153 TOWNSEND, SUITE 800, SAN FRANCISCO, CA, 94107, US

(21) Appl. No.: 10/893,608

(22) Filed: Jul. 15, 2004

Related U.S. Application Data
(60) Provisional application No. 60/487,360, filed on Jul. 15, 2003.

(51) Int. Cl. A61K 39/395

(52) U.S. Cl. 424/130.1

(57) ABSTRACT

The present invention provides methods and agents useful for the treatment of autoimmune conditions, especially primary biliary cirrhosis. The present invention also provides methods and kits useful for diagnosis or prognosis of primary biliary cirrhosis by detecting the presence of N. aromaticivorans. In addition, the present invention provides methods for identifying agents useful for the treatment of autoimmune conditions, especially primary biliary cirrhosis.

### CROSS REFERENCE TO RELATED APPLICATION(S)

This application claims the benefit of priority under 35 U.S.C. § 119(e) of U.S. Ser. No. 60/487,360 filed Jul. 15, 2003, the entire contents of which are incorporated herein by reference.

This invention was made in part with government support under Grant Nos. DK39588 and ES103019 awarded by the National Institutes of Health (NIH). The government may have certain rights in this invention.

### FIELD OF THE INVENTION

This invention relates generally to the field of autoimmune diseases, especially primary biliary cirrhosis.

### BACKGROUND OF THE INVENTION

Autoimmunity is characterized by production of either antibodies that react with host tissue or immune effector T cells that are autoreactive. Examples of clinically relevant autoantibodies are antibodies against acetylcholine receptors in myasthenia gravis; and anti-DNA, antierythrocyte, and antiplatelet antibodies in systemic lupus erythematosus.

Primary biliary cirrhosis (PBC), is an enigmatic autoimmune disease leading to destruction of intrahepatic bile ducts (Kaplan, MM, N Engl J Med, 335:1570-80 (1996)). The disease is progressive in nature, with a significant proportion of affected patients going on to develop cirrhosis with all its sequelae. PBC is frequently associated with a variety of disorders considered to be autoimmune in nature, such as the CREST syndrome (calcinosis, Raynaud's phenomenon, sclerodactyly, telangiectasia), the sicca syndrome (dry eyes and dry mouth), autoimmune thyroiditis, and renal tubular acidosis.

Females are more commonly affected, with approximately 90% of all cases of PBC occurring in females. This disease has been diagnosed in patients as young as 23 years and as old as 72. The majority of cases are diagnosed in the 40-60 age group. Thus far, only one childhood case of PBC has been reported. (Dahlan Y, Gastroenterology 125(5):1476-9, 2003).

Whilst patients suffering from PBC are at risk of developing cirrhosis, such patients are often profoundly symptomatic with significant impairment of their quality of life. The most common presenting symptom is pruritus or generalised itching. This may occur after the onset of birth control use or during pregnancy. Jaundice (yellowing of the eyes, skin and under the tongue) is seen as a later finding. More recently it has been appreciated that PBC patients can suffer from debilitating fatigue, indeed population surveys have suggested that fatigue may be the commonest symptom of PBC occurring in up to 80% of patients. The effects of PBC related fatigue on patient quality of life can be significant.

Other symptoms seen in PBC include bone and joint pains, abdominal pains and dry eyes and dry mouth from kerato-conjunctivitis sicca. Complications of PBC include portal hypertension, oesophageal varices, hepatic encephalopathy and osteomalacia.

Currently diagnosis of PBC is by routine blood tests, which reveal elevations in the blood cholesterol and alkaline phosphatase levels. Special serological tests directed to anti-mitochondrial antibodies help confirm the diagnosis. A circulating IgG antimitochondrial antibody is detected in more than 95 percent of patients with PBC and only rarely in other forms of liver disease. In addition, elevated serum levels of IgM and cryoproteins consisting of immune complexes capable of activating the alternate complement pathway are found in 80 to 90 percent of patients. The histologic finds of PBC patients resemble those noted in graft-versus-host disease following liver and bone marrow transplantation.

Patients who are without symptoms at the time of diagnosis have a better prognosis and can live 10 years or more, often without symptoms. Those who manifest symptoms of this disease, in contrast, have only a 50% survival rate beyond 5 years.

Both infectious and environmental agents have been proposed as immunological triggers for PBC. Recently, new strains of bacteria have been defined within the Sphingomonas genus and coined Novosphingobium spp.; these organisms are ubiquitous, metabolize complex organic chemical compounds, and convert environmental estrogens to native forms. Novosphingobium aromaticivorans, a gram-negative, strictly aerobe, has been recently defined and classified within the Sphingomonas genus (Takeuchi, M., et al., Int J Syst Evol Microbiol, 51:1405-17 (2001)), a family including a number of species sharing the ability to degrade polycyclic aromatic hydrocarbons (Shuttleworth, K L, et al., Mol Cells, 10:199-205 (2000); and Shi, T., et al., J Ind Microbiol Biotechnol, 26:283-9 (2001)).

There is a need in the art to provide methods and agents useful for treating autoimmune conditions, especially PBC. There is also a need in the art to provide methods and agents useful for diagnosis or prognosis of PBC.

### SUMMARY OF THE INVENTION

The present invention is based, in part, on the discovery that a gram-negative bacterial strain, e.g., N. aromaticivorans is involved in autoimmune conditions, especially primary biliary cirrhosis. Accordingly the present invention provides methods and agents useful for treating autoimmune conditions, e.g., primary biliary cirrhosis by inhibiting microbial activity, especially N. aromaticivorans. The present invention also provides methods and kits useful for diagnosis or prognosis of primary biliary corrhosis. In addition, the present invention provides screening methods for identifying agents useful for treating autoimmune conditions, especially primary biliary cirrhosis.

In one embodiment, the present invention provides a method of treating an autoimmune condition. The method comprises administering to a subject in need of such treatment an anti-microbial agent.

In another embodiment, the present invention provides a method for treating an autoimmune condition. The method comprises administering to a subject in need of such treatment an agent, wherein the agent specifically binds to a complex comprising MHC and a peptide from a protein having an amino acid sequence selected from the group consisting of SEQ ID NO. 1, SEQ ID NO. 2, SEQ ID NO. 3, and SEQ ID NO. 4.

In yet another embodiment, the present invention provides an antibody which specifically binds to N. aromaticivorans.

In yet another embodiment, the present invention provides an antibody which specifically binds to a protein with an amino acid sequence selected from the group consisting of SEQ ID NO. 1, SEQ ID NO. 2, SEQ ID NO. 3, and SEQ ID NO. 4, wherein the antibody does not specifically bind to an epitope of human PDC-E2.

In yet another embodiment, the present invention provides an antibody which specifically binds to a complex comprising an MHC molecule and a peptide from a protein having an amino acid sequence selected from the group consisting of SEQ ID NO. 1, SEQ ID NO. 2, SEQ ID NO. 3, and SEQ ID NO. 4, wherein the antibody does not specifically bind to an epitope of human PDC-E2.

In another embodiment, the present invention provides a method for diagnosing primary biliary cirrhosis in a subject. The method comprises determining the presence of N. aromaticivorans in a sample from a subject, wherein the presence of N. aromaticivorans in the sample is indicative of primary biliary cirrhosis in the subject.

In yet another embodiment, the present invention provides a kit useful for diagnosis or prognosis of primary biliary cirrhosis. The kit comprises an agent and an instruction, wherein the agent specifically detects the presence of N. aromaticivorans in a sample.

In still another embodiment, the present invention provides a method for screening for an agent useful for treating an autoimmune condition. The method comprises screening for an agent capable of binding to N. aromaticivorans or inhibiting the growth of N. aromaticivorans.

### SUMMARY OF THE FIGURES

FIG. 1 shows the amino acid alignment of the inner domain (aa 208-237), including the epitope recognized by B-cells, CD4⁺, and CD8⁺ cells, of human PDC-E2 with bacterial species.

### DETAILED DESCRIPTION OF THE INVENTION

The present invention is based, in part, on the discovery that microorganisms are associated with autoimmune conditions. In particular, certain bacterial strains, e.g., N. aromaticivorans, are associated with autoimmune conditions, especially primary biliary cirrhosis. Accordingly, the present invention provides methods of using anti-microbial agents for the treatment of autoimmune conditions. The present invention also provides methods and kits useful for diagnosis or prognosis of primary biliary cirrhosis by using agents capable of detecting the presence of N. aromaticivorans. In addition, the present invention provides agents useful for treating autoimmune conditions and screening methods useful for identifying these agents.

According to the present invention, an autoimmune condition can be treated by administering to a subject in need of such treatment an anti-microbial agent. The autoimmune condition can be any condition involving an autoimmune response, especially an autoimmune response associated with autoantigens including, without any limitation, mitochondrial autoantigens. In one embodiment, the autoimmune condition is associated with primary biliary cirrhosis. In another embodiment, the autoimmune condition is associated with a disease having an autoimmune response involving mitochondrial autoantigens such as human PDC-E2 and/or autoantibodies against mitochondrial autoantigens. In yet another embodiment, the autoimmune condition is associated with a disease having an autoimmune response identical or similar to the autoimmune response involved in primary biliary cirrhosis.

In still another embodiment, the autoimmune condition is associated with CRST syndrome (calcinosis, Raynaud's phenomenon, sclerodactyly, telangiectasia), the sicca syndrome (dry eyes and dry mouth), autoimmune thyroiditis, or renal tubular acidosis. In still yet another embodiment, the autoimmune condition is associated with ankylosing spondylitis, antiphospholipid syndrome, Crohn's disease, ulcerative colitis, insulin dependent diabetes, fibromyalgia, Goodpasture syndrome, Grave's disease, lupus, multiple sclerosis, myasthenia gravis, myositis, pemphigus vulgaris, rheumatoid arthritis, sarcoidosis, scleroderma, or Wegener's granulomatosis.

The anti-microbial agent used in the present invention can be any agent capable of decreasing or inhibiting the activity, e.g., the growth, of bacteria, especially gram-negative bacterial strains from the Sphingomonas genus, e.g., N. aromaticivorans. In one embodiment, the anti-microbial agent used in the present invention is an agent capable of decreasing or inhibiting the activity, e.g., the growth, of N. aromaticivorans or bacterial strains similar to N. aromaticivorans.

In general, bacterial strains similar to N. aromaticivorans are, for example, bacterial strains containing a homologue of human PDC-E2 and capable of metabolizing compounds containing hydrocarbons, e.g., halogenated hydrocarbons or polycyclic aromatic hydrocarbons. Bacterial strains containing a homologue of human PDC-E2 as referred to in the present invention include any bacteria containing a protein that is homologous to human PDC-E2 and the degree of homology between human PDC-E2 and the bacterial PDC-E2 is normally equal to or higher than the degree of homology found between human PDC-E2 and N. aromaticivorans PDC-E2.

In another embodiment, the anti-microbial agent used in the present invention is an antibiotic, e.g., Ciprofloxacin, erythromycin, gentamicin sulfate, naxcel (third generation cephalosporin), neomycin sulfate, novobiocin and polymyxin B.

In yet another embodiment, the anti-microbial agent used in the present invention is an antibody, e.g., an antibody against N. aromaticivorans or other bacterial strains similar to N. aromaticivorans. For example, the anti-microbial agent used in the present invention can be an antibody which specifically binds and/or causes damage to N. aromaticivorans or other bacterial strains similar to N. aromaticivorans, e.g., via the antibody itself, antibody-induced immune functions such as complement-mediated cytotoxicity or in combination with moieties operatively linked to the antibody including, without any limitation, toxic moiety, radio-activated moiety, and anti-microbial moiety, e.g., anti-microbial peptide, etc.

According to another aspect of the present invention, an autoimmune condition can also be treated by administering to a subject in need of such treatment an agent capable of binding to an MHC/peptide complex containing a peptide from a protein including PDC-E2 or homologues of PDC-E2. In general, the peptide in the MHC/peptide complex can be any length suitable, e.g., from 10 amino acids up to about 100, 200 or 300 amino acids for antigen presentation while the MHC molecule in the MHC/peptide complex can be an MHC class I molecule or an MHC class II molecule and should be the same origin as the subject to be treated by the agent, e.g., human MHC such as HLA if the subject is a human.

In one embodiment, the agent used in the present invention to treat an autoimmune condition is capable of specifically binding to a complex of MHC and a peptide from a protein containing N. aromaticivorans PDC-E2. In another embodiment, the agent used in the present invention is capable of specifically binding to a complex of MHC and a peptide from a protein having an amino acid sequence as shown in SEQ ID NO. 1, SEQ ID NO. 2, SEQ ID NO. 3, or SEQ ID NO. 4.

In yet another embodiment, the agent used in the present invention is a MHC/peptide monoclonal antibody, e.g., peptide-specific MHC-restricted monoclonal antibody capable of specifically binding to an MHC/peptide complex involved in antigen presentation of an antigen associated with N. aromaticivorans, especially an antigen of N. aromaticivorans PDC-E2. In still another embodiment, the agent used in the present invention is a MHC/peptide monoclonal antibody capable of inhibiting T cell proliferation, e.g., inhibiting the growth of CD4 and CD8 T cells in vitro or in vivo.

Any suitable method can be used to make such antibody. For example, such antibody can be produced by immunization of transgenic mice with soluble peptide/MHC complexes. In general, the recipient mice can be transgenic for, and partially tolerant to, the soluble MHC molecule complexed to its endogenous repertoire of peptides as described in Polakova et al., (The Journal of Immunology, 2000, 165: 5703-5712), which is incorporated herein by reference in its entirety. Alternatively, such antibody can be produced by immunization with APCs expressing an MHC molecule homogeneously loaded with a single peptide derived from a polypeptide containing PDC-E2 or homologues of PDC-E2, e.g., N. aromaticivorans PDC-E2. The general method is described in Zhong et al., (PNAS Vol. 94, pp. 13856-13861, December 1997, Immunology), which is incorporated herein by reference in its entirety.

Another feature of the present invention provides methods useful for diagnosis or prognosis of primary biliary cirrhosis. According to the present invention, primary biliary cirrhosis can be diagnosed by screening for the presence of N. aromaticivorans in a sample, e.g., a blood, sera, or biopsy sample from a subject, e.g., human. For example, the presence of N. aromaticivorans in a sample from a test subject indicates that the subject has primary biliary cirrhosis or conditions associated therewith.

In one embodiment, the presence of N. aromaticivorans can be used to detect primary biliary cirrhosis in subjects that are negative for antimitochondrial antibody or mitochondrial autoantigen, e.g., the presence of N. aromaticivorans and absence of antimitochondrial antibody or mitochondrial autoantigen in a test subject indicates that the subject has primary biliary cirrhosis or conditions associated therewith. In another embodiment, the presence of N. aromaticivorans in a sample from a subject indicates early stage primary biliary cirrhosis in the subject without the need for the subject to undergo liver biopsy to test for fibrosis or cirrhosis.

According to another aspect of the present invention, detection of the presence of N. aromaticivorans in a sample of a subject can predicate the development of primary biliary cirrhosis in the subject at a later stage, even if the subject does not yet exhibit any significant symptom of primary biliary cirrhosis or is free from any symptom of primary biliary cirrhosis.

In one embodiment, the presence of N. aromaticivorans in a sample is evaluated in combination with the subject's genetic susceptibility to primary biliary cirrhosis to predicate the subject's likelihood of developing primary biliary cirrhosis, e.g., a N. aromaticivorans positive subject who is also a relative of a primary biliary cirrhosis patient is more likely to develop primary biliary cirrhosis at a later stage than a subject who has no genetic relation to a patient with primary biliary cirrhosis.

In another embodiment, the presence of N. aromaticivorans in a sample is evaluated in combination with the subject's exposure to chemical contaminants in the environment, e.g., a N. aromaticivorans positive subject who has also been exposed to chemical contaminants is likely to develop primary biliary cirrhosis at a later stage than a subject who has not had such exposure.

According to the present invention, the presence of N. aromaticivorans can be detected by any suitable means known or later developed. For example, the presence of N. aromaticivorans can be identified by screening a test sample for N. aromaticivorans specific DNA, protein, metabolite, or enzymatic activity, or N. aromaticivorans specific host response, e.g., an antibody developed by a system such as human against N. aromaticivorans.

In one embodiment, the presence of N. aromaticivorans can be determined by detecting the presence of a nucleotide sequence of N. aromaticivorans via polymerase chain reaction including any amplification reaction, e.g., either thermocyling or isothermal amplification methods.

Isothermal amplification refers to a category of amplification in which amplification is carried out at a substantially constant temperature. Examples of isothermal amplification include transcription-mediated amplification (TMA), nucleic acid sequence based amplification (NASBA), strand-displacement amplifcation (SDA), rolling circle amplification (RCA), single primer isothermal amplification (SPIA™), and exponential single primer isothermal amplification (X-SPIA™), self-sustained sequence replication (3SR) and loop mediated isothermal amplification (LAMP). Various homogeneous real-time detection means can be used to detect the amplified products, including without any limitation, fluorescence polarization (FP), restriction endonuclease-mediated cleavage of a FRET probe, and molecular beacon, etc.

Thermacycling amplification methods such as PCR utilize two oligonucleotides, DNA polymerization components and a thermocycling machine to copy a specific sequence exponentially. Several closed homogeneous assay systems have been developed to detect amplified products generated by thermacycling amplification. One way is to use a nucleic acid intercalator which favorably binds to double-stranded DNA and yields higher fluorescence.

Recently a 5′ nuclease assay has been developed. In the 5′ nuclease assay, a third oligonucleotide, most common a fluorescense resonance energy transfer (FRET) probe containing a fluorophore and a quencher group, is included in PCR amplification reaction. Upon binding to target sequence, the probe is degraded by 5′ nuclease activity of certain DNA polymerases such as Taq and Tth DNA polymerase. Physical separation of fluorophore and quencher results in increased fluorescence. By monitoring change in fluorescence, presence of target sequence is detected qualitatively and quantitatively.

In another embodiment, the presence of N. aromaticivorans can be determined by detecting the presence of an amino acid sequence of N. aromaticivorans, e.g., an amino acid sequence specific to N. aromaticivorans and within or outside of N. aromaticivorans PDC-E2 region.

In yet another embodiment, the presence of N. aromaticivorans can be determined by detecting the presence of an amino acid sequence contained within SEQ ID NO. 1, SEQ ID NO. 2, SEQ ID NO. 3, or SEQ ID NO. 4, e.g., an amino acid sequence specific to N. aromaticivorans.

In still another embodiment, the presence of N. aromaticivorans can be determined by detecting a metabolite or an enzymatic activity that is specific to or characteristic of N. aromaticivorans. For example, the presence of N. aromaticivorans can be determined by detecting its activity of metabolizing compounds containing hydrocarbons, e.g., halogenated hydrocarbons or polycyclic aromatic hydrocarbons.

In still yet another embodiment, the presence of N. aromaticivorans can be determined by detecting any host response specific to N. aromaticivorans. For example, the presence of N. aromaticivorans can be determined by detecting any antibody against N. aromaticivorans generated by the host system, e.g., human.

Another feature of the present invention provides a kit useful for diagnosis or prognosis of primary biliary cirrhosis. According to the present invention, the diagnostic or prognostic kit provided by the present invention contains an agent capable of detecting the presence of N. aromaticivorans either directly or via a secondary reaction. For example, such agent can be any molecule capable of specifically interacting with N. aromaticivorans, while such interaction is detectable either directly or via a secondary reaction, e.g., enzymatic, radioactive, or any other reporting or signaling reaction.

In one embodiment, the agent used in the diagnostic or prognostic kit provided by the present invention is an antibody capable of specifically binding to N. aromaticivorans in a test sample, e.g., blood or serum sample. Such antibody can be optionally conjugated with a detectable or signaling entity or used in combination with a secondary agent for detection. For example, the diagnostic or prognostic kit provided by the present invention can contain a first antibody capable of specifically binding to N. aromaticivorans and a secondary reporting or detectable agent capable of detecting the first antibody, e.g., as it is bound to N. aromaticivorans or the binding of the first antibody to N. aromaticivorans.

In another embodiment, the diagnostic or prognostic kit provided by the present invention contains one or more oligonucleotides useful for amplifying and/or detecting nucleotide sequences of N. aromaticivorans. Such oligonucleotides can be any oligonucleotide suitable for thermocycling or isothermal amplification reactions designed to amplify a nucleotide sequence of N. aromaticivorans. For example, such oligonucleotides can contain a nucleotide sequence specific to N. aromaticivorans and optionally a detectable moiety for fluorescence polarization, restriction endonuclease-mediated cleavage of a FRET probe, and molecular beacon.

According to another feature of the present invention, agents useful for treating an autoimmune condition, especially primary biliary cirrhosis, can be identified by screening for an agent capable of binding to N. aromaticivorans, N. aromaticivorans PDC-E2, an MHC/peptide complex containing a peptide from N. aromaticivorans PDC-E2 or homologues thereof, or inhibiting the growth of N. aromaticivorans, e.g., killing N. aromaticivorans.

The present invention also provides antibodies against N. aromaticivorans. According to the present invention, the antibody provided by the present invention can be polyclonal or monoclonal antibody that specifically binds to N. aromaticivorans, or any epitope of N. aromaticivorans or N. aromaticivorans PDC-E2.

In one embodiment, the antibody provided by the present invention is an antibody capable of specifically binding to a protein having an amino acid sequence as shown in SEQ ID NO. 1, SEQ ID NO. 2, SEQ ID NO. 3, or SEQ ID NO. 4 or an epitope contained therein. In another embodiment, the antibody provided by the present invention is an antibody capable of specifically binding to N. aromaticivorans PDC-E2 or an epitope contained therein, but not substantially binding to other proteins, e.g., human or other PDC-E2 homologues or epitopes contained therein.

The antibody provided by the present invention can also be a peptide-specific MHC-restricted monoclonal antibody capable of binding to an MHC/peptide complex containing a peptide from N. aromaticivorans, especially N. aromaticivorans PDC-E2, e.g., as shown in SEQ ID NO. 1, SEQ ID NO. 2, SEQ ID NO. 3, or SEQ ID NO. 4. In one embodiment, the antibody provided by the present invention is capable of competing with αβ TCRs, e.g., blocking responses associated with αβ TCRs such as CTL priming.

The antibodies provided by the present invention usually can be used for therapeutic or diagnostic purposes. These antibodies or the agents used in the present invention can be provided as a composition, e.g., pharmaceutical composition including one or more antibodies or agents of the present invention and a carrier, e.g., pharmaceutically acceptable carrier. Pharmaceutically acceptable carriers are well known to those in the art. Such carriers include, without limitation, large, slowly metabolized macromolecules, e.g., proteins, polysaccharides, polylactic acids, polyglycolic acids, polymeric amino acids, amino acid copolymers, and inactive virus particles.

Pharmaceutically acceptable salts can also be used in the composition, for example, mineral salts such as hydrochlorides, hydrobromides, phosphates, or sulfates, as well as the salts of organic acids such as acetates, propionates, malonates, or benzoates. The composition can also contain liquids, e.g., water, saline, glycerol, and ethanol, as well as substances, e.g., wetting agents, emulsifying agents, or pH buffering agents. In addition, liposomes or other delivery particles can also be used as a carrier for the compositions of the present invention.

Typically, the antibodies or agents in the present invention useful for therapeutic treatment are prepared or formulated in general as an injectable, either as a liquid solution or suspension. However, solid forms suitable for solution in, or suspension in, liquid vehicles prior to injection can also be prepared. The antibodies or agents of the present invention can also be formulated into an enteric-coated tablet or gel capsule according to known methods in the art.

The antibodies or agents in the present invention useful for therapeutic treatment can be administered alone, in a composition with a suitable pharmaceutical carrier, or in combination with other therapeutic agents. An effective amount of the antibodies or agents of the present invention to be administered can be determined on a case-by-case basis. Factors should be considered usually include age, body weight, stage of the condition, other disease conditions, duration of the treatment, and the response to the initial treatment.

The antibodies or agents of the present invention may be administered in any way which is medically acceptable which may depend on the disease condition or injury being treated. Possible administration routes include injections, by parenteral routes such as intravascular, intravenous, intraepidural or others, as well as oral, nasal, ophthalmic, rectal, topical, or pulmonary, e.g., by inhalation. The antibodies or agents of the present invention may also be directly applied to tissue surfaces, e.g., during surgery. Sustained release administration is also specifically included in the invention, by such means as depot injections or erodible implants.

### EXAMPLES

The following examples are intended to illustrate but not to limit the invention in any manner, shape, or form, either explicitly or implicitly. While they are typical of those that might be used, other procedures, methodologies, or techniques known to those skilled in the art may alternatively be used.

### Introduction

Among potential pathogens involved in molecular mimicry, bacterial infections, particularly due to E. coli, have been extensively investigated (Van de Water, J., et al., Hepatology, 33:771-5 (2001)). Thus, cross-reactivity of antimitochondrial antibodies (AMA) with epitopes present in enzymes involved in the bacterial respiratory chain or in other peptides has been described for E. coli (Fussey, S P, et al., Proc Natl Acad Sci USA, 87:3987-91 (1990); Fussy, S P, et al, Hepatology, 13:467-74 (1991); Bogdanos, D P, et al., Liver, 21:225-32 (2001); and Mayo, I., et al., J Hepatol, 33:528-36 (2000)), and Lactobacillus delbrueckii (Bogdanos, D P, et al., Hepatology, 32:300A (2000)), among others. These organisms were studied because proteins in these bacteria have homology with the E2 component of human pyruvate dehydrogenase complex (PDC-E2), the major autoantigen in PBC. However, the resulting data do not support a causal connection to PBC as titers to bacterial PDC-E2 were lower and appeared later in the disease than responses to self (human PDC) (Fussey, S P, et al., Hepatology, 13:467-74 (1991)). Another etiologic factor that has received attention is that of xenobiotic exposure (Long, S A, et al., J Immunol, 167:2956-63 (2001)).

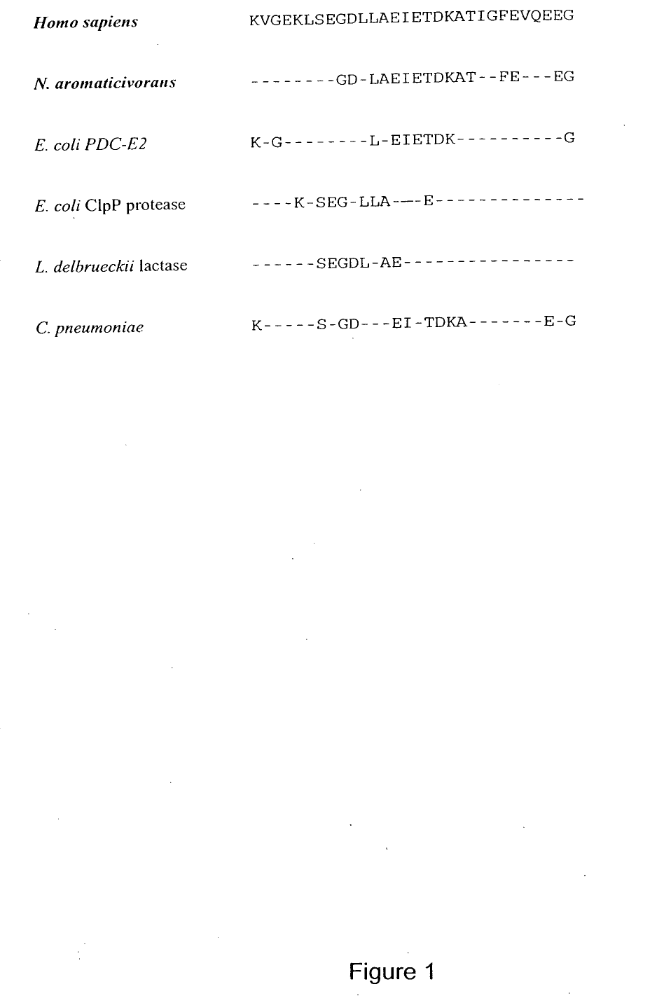

The amino acid sequences of two proteins of N. aromaticivorans have a high degree of homology with the dominant immunogenic lipoylated domain (amino acid positions 208-237) of the E2 subunit of human PDC (FIG. 1). This is the highest level of homology for any known microorganism and this mitochondrial autoantigen. This microorganism is environmentally ubiquitous and found in soil, water and coastal plain sediments (Takeuchi, M., et al., Int J Syst Evol Microbiol, 51:1405-17 (2001)). Although the list of degradable chemicals is far from being complete, N. aromaticivorans is unique because of the potential to be utilized in the bioremediation of wastage (Frederickson, J K, et al., Appl Environ Microbiol, 61:1917-22 (1995)).

Importantly, a bacterial strain from the Sphingomonas genus showing 96% DNA homology with N. aromaticivorans presented cleavage activity on 17β-estradiol, transforming the inactive conjugated to the free active form (Fujii, K., et al., Appl Environ Microbiol, 68:2057-60 (2002)). While other strains from the same genus have been shown to cause human disease (Lemaitre, D., et al., J Hosp Infect, 32:199-206 (1996); and Perola, O., et al., J Hosp Infect, 50:196-201 (2002)), no pathogenic effect has to date been associated with the strain described herein.

We investigated sera reactivity of patients with PBC and controls against N. aromaticivorans. We report a highly specific and directed response that is found only in PBC. Thus this bacterium, alone or in combination with chemical contaminants, initiates molecular mimicry and the development of PBC in genetically susceptible hosts.

### Example 1

### Materials and Methods

Subjects

Serum samples from a total of 97 randomly selected, well-characterized patients with PBC, 46 first-degree female relatives (mothers, sisters and daughters) and 10 spouses (husbands) of patients, were studied. In addition, there were 195 control subjects, including 49 with primary sclerosing cholangitis (PSC), 10 with rheumatoid arthritis (RA), 12 with autoimmune hepatitis (A1H), and 124 healthy subjects.

The diagnosis of PBC (Kaplan, MM, N Engl J Med, 335:1570-80 (1996)), PSC (Lee, Y M, et al., Am J Gastroenterol, 97:528-34 (2002)), RA (Hakala, M., et al., J Rheumatol, 20:1674-8 (1993), and AIH (Johnson, P J, et al., Hepatology, 18:998-1005 (1993)) were based on internationally accepted criteria. All patients' sera were obtained from the PBC center at the University of Milan (Ospedale San Paolo). Approximately half of the healthy controls (64/124) were sex, age and geographically-matched to the Milan PBC population. As an additional control, the remaining healthy sera (60/124) were obtained from an age and sex-matched population in northern California. Finally, a nested substudy was also performed on 20 PBC patients from the western United States.

All sera were examined for reactivity against mitochondrial autoantigens as well as protein lysates of N. aromaticivorans and E. coli. We also note that all PBC patients were known to be negative for hepatitis B surface antigen and antibodies against hepatitis C virus. Further, all patients had undergone liver biopsy within the previous 18 months. The patients with no fibrosis at liver biopsy, i.e. those with stage I and II, according to Ludwig et al. (Ludwig, J., et al., Virchows Arch A Pathol Anat Histol, 379:103-12 (1978)), were considered to have early-stage disease (n=46); patients with fibrosis or cirrhosis, i.e. stage III or IV, or those with a history of major complications from liver cirrhosis (i.e. ascites requiring diuretic therapy, gastrointestinal bleeding due to portal hypertension, hepatic encephalopathy, or hepatocellular carcinoma) were considered to have advanced disease (n=51).

Sixty-seven patients (69%) among all patients and 37 (73%) among patients with advanced disease, were currently receiving ursodeoxycholic acid as the only treatment for PBC at the time of blood sampling. The study protocol follows the ethical guidelines of the 1975 Declaration of Helsinki and subsequent modifications.

AMA Specificities

The AMA status of PBC patients was investigated by ELISA using recombinant PDC-E2, oxoglutaric dehydrogenase complex (OGDC-E2), and branched-chain α-ketoacid dehydrogenase complex (BCOADC-E2) as previously described (Miyakawa, H., et al., Hepatology, 34:243-8 (2001)). Sera reacting against one or more of these antigens were considered to be AMA-positive.

Bacteria

Bacterial cultures of N. aromaticivorans were used to streak trypticase soy agar plates which were incubated at 37° C. for 24 hours. Single colonies were isolated from this plate and used to inoculate 2.0 ml trypticase soy broth (TSB) which were then incubated at 37° C. for 24 hours. One ml of these cultures was used to inoculate 50 ml TSB cultures grown for 24 hours on an orbital shaker at 37° C. E. coli strains were grown at 37° C. in L broth.

Fifty ml of a 24-hour culture were centrifuged at 10,000 g for 1 hour. The supernatant fluid was discarded and the bacterial pellet dissolved in 5 ml of a 10 mM Tris, 0.15 M NaCl, 0.05% Tween-20 with a protease inhibitor cocktail (Complete, Boehringer Mannheim, Ingelheim, Germany). The suspension was freeze-thawed 3 times and sonicated. The suspension was then centrifuged at 20,000 g for 1 hour. The supernatant fluid was collected, aliquoted, and stored at −80° C.

Immunoblotting Against Bacterial Antigens

Two hundred μg of proteins from a bacterial lysate were loaded onto a 10% SDS PAGE gel (10×10 cm) and electrophoresed at 20 mA for 2 hours. Proteins were transferred onto nitrocellulose membranes and blocked with 5% powdered milk. Membrane strips were incubated with sera diluted 10⁻³ through 10⁻⁶. After washing, strips were incubated with a horseradish peroxidase (HRP)-conjugated goat anti-human Ig (Zymed Laboratories Inc., South San Francisco, Calif.) for 1 hour. The washed blots were developed using Super Signal Substrate (Pierce, Rockford, Ill.) and exposed to X-ray film.

Specificity of Reactivity

Firstly, two different human PDC-E2 specific mouse-derived IgG monoclonal antibodies (clones 4C8 and 1F1) (Miyakawa, H., et al., Hepatology, 34:243-8 (2001); and Migliaccio, C., et al., J Immunol, 161:5157-63 (1998)), and rabbit anti-lipoic acid antisera (Sasaki, M., et al., J Autoimmun, 15:51-60 (2000)) were studied by Western blots using HRP-conjugated goat anti-mouse or anti-rabbit IgG (Zymed Laboratories Inc.) at a 10⁻⁴ dilution as secondary antibody.

Second, optimally diluted PBC sera were incubated with immobilized recombinant PDC-E2, or an irrelevant control, for 14 hours at 4° C. The absorbed sera were then centrifuged at 2,500 g for 15 minutes and subsequently used as probes.

Third, purified recombinant PDC-E2 (100 μg) was separated on a 10% SDS-PAGE gel and transferred to nitrocellulose. The membrane was then cut into strips containing separate proteins and the strips blocked with 5% milk for 1 hour before incubation with sera diluted at 1:100. The strips were washed and subjected to elution with 0.1 M glycine-HCl, 20 mM MgCl₂, 50 mM KCl, and 0.5% Tween 20 at pH 2.5. The eluate was adjusted to neutral pH using 1 M Tris. Known positive and negative sera were included as controls throughout the study.

### Example 2

### Sera from Patients with PBC React in a Highly Directed and Specific Fashion Against Proteins from N. Aromaticivorans.

A genome blast search of N. aromaticivorans sequences using the inner domain protein sequence of human PDC-E2 (KVGEKLSEGDLLAEIETDKATIGFEVQEEG) revealed 4 potential lipoylated proteins with molecular weights of 50.1, 48.9, 47.3 and 42.5 kDa (GenBank access of contigs NZ-AAAV01000159, NZ-AAAV01000132, and NZ-AAAV010000146). The lipoation was confirmed using anti-lipoic acid specific rabbit antisera. Homology and domain searches confirmed that these proteins belong to the 2-oxoacid dehydrogenase complex family. Two of these proteins have significant homology to the inner lipoyl domain of human PDC-E2 (FIG. 1).

Patients with PBC were classified by AMA status using recombinant mitochondrial autoantigens and ELISA, since these antigens have been shown to be the most sensitive and specific for the detection of AMA(Miyakawa, H., et al., Hepatology, 34:243-8 (2001)). Of the 97 patients with PBC, there were 80 patients who were AMA positive and 17 that were AMA negative (Table 1).

TABLE 1
Reactivities against 47 and 50 KDa proteins from
N. aromaticivorans in sera from PBC patients
according to AMA status and in sera from control populations.

| | Anti-N. aromaticivorans |
|---|---|
| | positive |
| AMA-positive PBC (n = 80) | |
| Anti PCD-E2 pos (n = 77) | 77 (100%)* |
| Anti PDC-E2 pos only (n = 43) | 43 (100%)* |
| Anti BCOADC-E2 pos only (n = 3) | 1 (33%) |
| Anti PDC-E2 and OGDC pos (n = 2) | 2 (100%) |
| Anti PDC-E2 and BCOADC-E2 pos (n = 28) | 28 (100%)* |
| Anti PDC-E2, OGDC, and BCOADC-E2 pos | 4 (100%) |
| (n = 4) | |
| AMA-negative PBC (n = 17) | 2 (12%) |
| PBC relatives (n = 46) | 1 (2%)** |
| PBC spouses (n = 10) | 0 |
| Primary sclerosing cholangitis (n = 49) | 0 |
| Autoimmune hepatitis (AIH) (n = 12) | 0 |
| Rheumatoid arthritis (n = 10) | 0 |
| Healthy subjects (n = 124)*** | 0 |

*P < 0.001 compared to the control groups
**This relative, a sister of a patient with PBC, reacted to the bacterium while clinically asymptomatic; two years later was independently diagnosed with PBC.
***Healthy controls include sex-matched subjects from US (n = 60) and Italy (n = 64)

One hundred percent of the PBC patients that had antibodies to PDC-E2 (77/77) reacted with two proteins (molecular weight 47 and 50 kDa) from N. aromaticivorans. There were three patients who were AMA positive but only had antibodies that reacted against BCOADC-E2. Of these three patients, ⅓ reacted with N. aromaticivorans. There were 17 AMA negative patients with PBC; 2/17, or 12% of the sera, despite being AMA negative by immunoblotting, reacted to N. aromaticivorans.

Forty-six first-degree relatives of patients with PBC were studied and, importantly, one 51-year old female (considered healthy) was found to be both anti-PDC-E2 positive and to have antibodies to N. aromaticivorans. This patient, two years after serum collection, developed PBC. In contrast, none of the 195 control sera had antibodies to N. aromaticivorans (Table 1). These two protein bands correspond to the gene bank identification of two forms of PDC-E2 in the N. aromaticivorans (FIG. 1). Finally, the nested substudy of U.S. PBC patients provided the same results against the bacterium as the Italian sera.

Antibodies against N. aromaticivorans were found in early and late stage PBC patients (Table 2).

TABLE 2
Reactivities against N. aromaticivorans proteins
(47 and 50 Kda proteins) in sera from PBC patients
according to histological stage and AMA status.

| | Anti-N. aromaticivorans |
|---|---|
| | positive |
| Early-stage disease (n = 46) | |
| AMA positive (n = 38) | |
| All anti PDC-E2 pos (n = 36) | 36 (100%) |
| Anti PDC-E2 pos only (n = 21) | 21 (100%) |
| Anti BCOADC-E2 pos only (n = 2) | 1 (50%) |
| Anti PDC-E2 and OGDC pos (n = 1) | 1 (100%) |
| Anti PDC-E2 and BCOADC-E2 pos (n = 13) | 13 (100%) |
| Anti PDC-E2, OGDC, and BCOADC-E2 pos | 1 (100%) |
| (n = 1) | |
| AMA negative (n = 8) | 1 (13%) |
| Advanced-stage disease (n = 51) | |
| AMA positive (n = 42) | |
| All anti PDC-E2 pos (n = 41) | 41 (100%) |
| Anti PDC-E2 pos only (n = 22) | 22 (100%) |
| Anti BCOADC-E2 pos only (n = 1) | 0 |
| Anti PDC-E2 and OGDC pos (n = 1) | 1 (100%) |
| Anti PDC-E2 and BCOADC-E2 pos (n = 15) | 15 (100%) |
| Anti PDC-E2, OGDC, and BCOADC-E2 pos | 3 (100%) |
| (n = 3) | |
| AMA negative (n = 9) | 1 (11%) |

More importantly, marked differences in the titers of reactivity of sera from PBC patients were observed when probed against N. aromaticivorans and E. coli. For example, lysates of N. aromaticivorans and E. coli were separated by PAGE, transferred to nitrocellulose and probed with a representative serum from a PDC-E2 positive patient with PBC at serial dilutions of 10⁻³ to 10⁻⁶. The intense degree of reactivity against proteins of 47 and 50 kD in N. aromaticivorans, corresponding to the two known PDC-like molecules in this species. In contrast, PDC-E2 in E. coli shown at 66 kD failed to react with serum at dilutions over 10^{-4.}

While 77/77 (100%) of the PBC sera reacted against N. aromaticivorans and 56/77 (73%) against E. coli at 10⁻³ dilution, the reactivity of the same sera dropped to 74/77 (96%) and 43/77 (56%) against N. aromaticivorans at 10⁻⁴ and 10⁻⁵, respectively, and to 19/77 (25%) and 1/77 (1%) against E. coli at the same dilutions. Indeed, even at 10⁻⁶, there were still 18 of 77, or 23% of patients that were reactive to N. aromaticivorans (Table 3).

TABLE 3
Serial dilutions of anti-PDC-E2-positive PBC sera*
against N. aromaticivorans and E. coli.

| | 10⁻³ | 10⁻⁴ | 10⁻⁵ | 10⁻⁶ |
|---|---|---|---|---|
| N. aromaticivorans** | 77/77 | 74/77 (96%) | 43/77 | 18/77 |
| | (100%) | | (56%) | (23%) |
| E. coli** | 56/77 (73%) | 19/77 (25%) | 1/77 | 0/77 |
| | | | (1%) | |

*Amongst sera that were anti-BCOADC-E2 positive only, 1/3 and 0/3 reacted at 10⁻⁴ against N. aromaticivorans and E. coli, respectively. Amongst AMA-negative sera that reacted with N. aromaticivorans, 2/2 reacted at 10⁻³ and 0/2 reacted at 10⁻⁴; 0/2 reacted against E. coli at all dilutions.
**P < 0.001 at all dilutions

Previously well-defined monoclonal antibodies, 4C8 and 1F1 (Miyakawa, H., et al., Hepatology, 34:243-8 (2001); and Migliaccio, C., et al., J Immunol, 161:5157-63 (1998)), whose specificity is only to PDC-E2, reacted to the same bands at 47 and 50 kd, similar to that seen in the sera from patients with PBC. For example, lysates of N. aromaticivorans were separated by PAGE, transferred to nitrocellulose, and probed with either a representative PBC sera at 1:1,000, affinity purified antibodies against recombinant PDC-E2, serum from a PBC patient following absorption with recombinant PDC-E2, and two monoclonal antibodies with specificity for PDC-E2. When sera from patients with PBC were absorbed against recombinant PDC-E2, the reactivity to N. aromaticivorans was lost. Lastly, affinity purified antisera to PDC-E2 also reacted to the same proteins of N. aromaticivorans.

### Example 3

### Cloning and Expression of N. Aromaticivorans Proteins

Four N. aromaticivorans lipoylated proteins (Novo 1-4) were cloned into either the expression plasmids pRSET (Invitrogen) or pCal-n-Flag (Stratagene) and expressed in E. coli. Briefly, genomic DNA was isolated from N. aromaticivorans and subjected to PCR amplification using specific primers for Novo 1-4 (Table 4).

TABLE 4

Primer sequences for PCR amplification of lipoylated
N. aromaticivorans proteins (Novo 1-4).

| | | |
|---|---|---|
| Protein | Forward primer | Reverse primer |

| | | |
|---|---|---|
| Novo 1 | AATAGGATCCATGCGCTGCGACATGGCG | AATAAAGCTTCAGGCGACCAGGCCGAGCG |
| | (SEQ ID NO. 5) | (SEQ ID NO. 6) |
| Novo 2 | AATAGGATCCATGGCAATCGAACTGAAG | AAAAAAGCTTCAGCGATAGCAGACCTTG |
| | (SEQ ID NO. 7) | (SEQ ID NO. 8) |
| Novo 3 | AAAGGATCCGTGCTGAACGAACTGCGCATC | AAAGAATTCTCACGGCCGTTCCAGCGTTG |
| | (SEQ ID NO. 9) | (SEQ ID NO. 10) |
| Novo 4 | AAATGGATCCATGGGAACCTACACATTCC | AAATGAATTCTCAGTCCGCCAGCAGCAGCAC |
| | (SEQ ID NO. 11) | (SEQ ID NO. 12) |

PCR products were subcloned into TOPO vectors (Invitrogen) and sequenced. Plasmids with the correct reading frame were then subcloned into expression vectors and transformed into E. coli BL21 (DE3) for recombinant protein expression. Recombinant proteins of Novo 1, 2, 3, and 4 have been used to conduct antibody reactivity against sera from patients with PBC and control. PBC sera recognized recombinant Novo 1, 2, 3, and 4 while sera from healthy controls do not react. It is noted that anti-His antibody has also recognized Novo 1, 2, 3, and 4.
Amino Acid Sequence Analysis of Novo 1-4

The deduced amino acid sequence of recombinant Novo 1 to 4 are shown below. The TDK residues of the lipoyl domains are underlined.

Novo1 (SEQ ID NO. 1)
MRCDMAICPSRSTGIGKFVCGASGNPLDPTRHAGEMAPRKDAGEVSATAPSSLRYQDTEN
TPMPIAIKMPALSPTMEEGTLAKWLVKVGDKVSSGDIMAEIETDKATMEFEAVDEGTIVS
IDVAEGSEGVKVGTVIATLAGEDEDASAPAPKAVAPAAAPVPVPAPKAEPAPAAVSTPAP
AAASASKGDRVIATPLAKRIAADKGIDLKGVAGSGPNGRIIRADVEGAKPAAAAPVSTVA
PAVASAAAPARAPAAVPDFGIPYEAQKLNNVRKTIARRLTEAKQTIPHIYLTVDIRLDAL
LKLRGDLNKALEAQGVKLSVNDLIIKALAKALMQVPKCNVSFAGDELRSFKRADISVAVA
APSGLITPIIVDAGSKSVSAIATEMKALANKAREGKLQPHEYQGGTASLSNLGMFSIKNF
DAVINPPQAMIMAVGAGEQRPYVIDGALGIATVMSATGSFDHRAIDGADGAELMQAFKNL
IENPLGLVA
Novo2 (SEQ ID NO. 2)
MAIELKMPALSPTMEEGTLAKWLVKAGDEVRSGDILAEIETDKATMEFEAVDEGVIAEIL
VAEGTEGVKVGTVIATIQGEGEDAAPAAATPAVEQKVEMSEAAPSVEARAAPAVAIAPKV
DAKPAVDPEIPAGTAMVPTTVREALRDAMAEEMRADDRVFVMGEEVAEYQGAYKVTQGLL
DEFGPRRVIDTPITEYGFVGIGAGAAMGGLRPIIEFMTFNFAMQAIDHIINSAAKTNYMS
GGQMRCPIVFRGPNGAASRVGAQHSQNYGPWYANVPGLVVIAPYDSADAKGLMKAAIRSE
DPVVFLENELVYGRTFDVPQMDDFVLPIGKARIVRQGKDVTIVSYSIGVGLALEAAETLA
AEGIDAEVIDLRTLRPLDKDTVLASLAKTNRLVVAEEGFPVCSIASEIMAICMEDGFDHL
DAPVLRVCDEDVPLPYAANLEKAALIDAGKIAAAVRKVCYR
Novo3 (SEQ ID NO. 3)
VLNELRIPRMGSVENARLLNWRVQEGEAYEPGQVLYEIETDKTSVEVEAEGPGVLARHLA
AVGDEFKVGDRIGLWALPGTAPATLRAALSPQPMPASEPAPSPSSTLPAAVSAPGLHALR
PVSRDAAGGRRVSPLARRLAAQNGVDLATVTGTGMGGKISGKDVLAASAKPRPAPVPVSP
PRPGSDGEIVPHSLRRRTIAQRMVEAAAIPTLTADMEVDLTALFARRRSVEGNGASVLGM
IAEAAIAALLQHRRLNAHWREDAMVQFGAVHLGIAVDTPEGLVVPVVRNAESLNARGLTD
AIAALADKARAGTLRPQDMEGGTFTISNPGSMGPVVRAEALLNPPQVALLGLPGIVRAPV
AIKDGDAWAMAVRPLLRLSLSFDHRALDGGPVIAFLNTLKATLERP
Novo4 (SEQ ID NO. 4)
MGTYTFRLPDIGEGIAEAEIVAWHVKVGDTVEEDGRLADMMTDKATVEMESPVAGKVVSV
AGEVGDVVAIGSALVVIETEGEDEAPAPAAAPAPKAAIVEERIEVETPEPPQPPSPPQPL
FVSREVEAPPAVPATGSGVAPGPRASTAPDTIGGAGAKVLASPAVRQRARDLGIDLSEVR
PSEEGRIRHADLDQFLSYNASGGYRAAGAERGDEVIRVIGMRRRIAENMAASKRHIPHFS
YVEECDVTALEIMREQLNAGRGDKPKLTMLPLLITAICRALPQYPMINARYDDEAGVVTR
YGAVHLGMAAQTPAGLMVPVIRNAQTLNLWQLAREIVRLAEAARSGSAKSDELSGSTLTV
TSLGPLGGVATTPVINRPEVAIIGPNRIVERPMFVSDGMGGERIEKRKLMNISISCDHRV
VDGHDAASFIQAVKKLIETPVLLLAD

Amino acid sequence comparison of Novo 1-4 and the human 2-oxo-acid dehydrogenase complex demonstrated significant sequence identity between Novo 1-4 and the specific 2-oxo-acid dehydrogenases. (Table 5).

TABLE 5

Molecular identity of Novo1-4 as determined by
amino acid sequence comparison.

| | | | |
|---|---|---|---|
| | Molecular | Sequence comparison with | % |
| Protein | Weight | 2-oxo-acid dehydrogenases | identity |

| | | | | | |
|---|---|---|---|---|---|
| Novo 1 | 50 kDa | PDC-E2 | GDLLAEIETDKATI | (SEQ ID NO. 13) | 78.6 |
| | | Novo 1 | GDIMAEIETDKATM | (SEQ ID NO. 14) | |
| Novo 2 | 49 kDa | PDC E2 | LAEIETDKATIGFEVQEEG | (SEQ ID NO. 15) | 73.6 |
| | | Novo 2 | LAEIETDKATMEFEAVDEG | (SEQ ID NO. 16) | |
| | | PDC E1β | LWKKYGDKRIIDTPISEMG | (SEQ ID NO. 17) | 21.1 |
| Novo 3 | 47 kDa | OGDC E2 | DEVVCEIETDKTSV | (SEQ ID NO. 18) | 71.4 |
| | | Novo 3 | GQVLYEIETDKTSV | (SEQ ID NO. 19) | |
| Novo 4 | 42 kDa | BCOADC-E2 | VQSDKASVTITSRYDG | (SEQ ID NO. 20) | 37.5 |
| | | Novo 4 | MMTDKATVEMESPVAG | (SEQ ID NO. 21) | |

In summary, we have cloned the four genes by PCR, involving multiple constructs and sequencing in both directions. The resulting contiguous were assembled and translated into corresponding primary amino acid sequences. Novo 1 is a 489 aa polypeptide homologous to PDC-E2, using the NCBI blast engine. The Novo 2 polypeptide contains 461 residues and homology to PDC-E2 and PDC E1 beta. Novo 3 contains 406 amino acids and homology to human OGDC-E2. Finally, Novo 4 contains 446 residues with homology to both human BCOADC-E2 and PDC-E2. All four cloned Novo proteins contained a single biotin-lipoyl domain, located at the amino terminal portion of the sequence, similar to human PDC-E2.

Specific Recognition of Recombinant Lipoylated N. Aromaticivorans Proteins by PBC Sera

Recombinant Novo proteins were purified by a Nickle column and studied for immune reactivity. Briefly, recombinant proteins of Novo 2 and Novo 4 were resolved by SDS-PAGE, transferred to nitrocellulose membranes and probed with sera. Of the AMA positive sera, 85.1% recognized wither Novo2 or Novo 4; with 78% recognized Novo 2 and 72% recognized Novo 4. Of the true AMA negative sera, 4.1% recognized Novo2 and 15.3% recognized Novo 4; with 15.3% sera reacted to either Novo 2 or Novo 4 (Table 6). Control sera did not react.

It should be noted that immunoblotting data of PBC sera against N. aromaticivorans lysate demonstrated that both AMA positive and AMA negative PBC sera reacted to Novo 1. Historically, AMAs were performed by immunofluorescence (IMF) using tissue substrates. At the level of the research bench, this changed dramatically when the mitochondrial autoantigens were cloned/identified and the subsequent use of ELISA and/or blotting with recombinant antigens. At this point it became clear that up to 50% of IMF AMA negative sera, in patients who were clinically diagnosed with PBC, were in fact AMA positive when recombinant antigens were used as substrates.

We emphasize this point because the AMA negative sera used herein are in patients who are clinically diagnosed with PBC, but are the important subgroup of patients who remain AMA negative whether tested by IMF or by blotting/ELISA with recombinant antigens. AMA negative PBC patients are similar to AMA positive PBC in their clinical biochemical and histological spectrum of the disease.

TABLE 6
Immunoreactivity of AMA positive PBC, AMA negative and healthy controls
against recombinant Novo 2 and Novo 4 proteins. ^{#} Determined at 1:10⁻⁴ sera
dilution by immunoblot; ^{##} determined at 1:10⁻³ sera dilution by immunoblot.

| | Reactivity against | | | |
|---|---|---|---|---|
| Sample | Mitochondrial | | | NOVO 2 or |
| Group | Antigens | NOVO 2^{#} | NOVO 4^{##} | NOVO 4 |
| AMA | PDC-E2 | 114/129 (88.4%) | 86/129 (66.7%) | 116/129 (89.9%) |
| Positive | BCOADC-E2 | 3/32 (9.4%) | 15/32 (46.9%) | 15/32 (46.9%) |
| (n = 328) | OGDC-E2 | 0/0 | 0/0 | 0/0 |
| | PDC-E2/ | 94/109 (86.2%) | 95/109 (87.1%) | 100/109 (91.7%) |
| | BCOADC-E2 | | | |
| | PDC-E2/OGDC- | 26/30 (86.7%) | 23/30 (76.7%) | 28/30 (93.3%) |
| | E2 | | | |
| | BCOADC-E2/ | 0/4 (0%) | 0/4 (0%) | 0/4 (0%) |
| | OGDC-E2 | | | |
| | PDC-E2/ | 19/24 (79.2%) | 20/24 (83.3%) | 20/24 (83.3%) |
| | BCOADC-E2/ | | | |
| | OGDC-E2 | | | |

| | | | |
|---|---|---|---|
| a. Number of | 256/328 (78%) | 239/328 (72%) | 279/328 (85.1%) |
| Reactive AMA positive sera | | | |
| b. ^{ρ}AMA | 3/72 (4.1%) | 11/72 (15.3%) | 11/72 (15.3%) |
| Negative (n = 72)* | | | |
| c. Healthy | 0/215 (0%) | 0/215 (0%) | 0/215 (0%) |
| Controls (n = 215)^{σ} | | | |

*Includes reactivity only to Novo 2 or Novo 4; data from lysates of N. aromaticivorans is presented above.
^{σ}Includes 195 healthy control sera probed against N. aromaticivorans lysates (4) and 20 healthy control sera probed against the recombinant Novo proteins. We have also probed Novo 1, 2 and 4 with sera from patients with PSC (N = 20) or SLE (N = 20) and all were non-reactive.
^{ρ}The collection of true AMA negative sera were derived from several sources in order to enrich the available sera for study.

The data reported herein demonstrate for the first time that sera from patients with PBC react in a highly directed and specific fashion against proteins from N. aromaticivorans. Further, this reactivity is at least 100-fold higher when compared to reactivity against E. coli, found in early stage patients, including 12% of AMA-negative patients, and can be detected even at 10⁻⁶ dilution.

This organism is of particular interest to environmental scientists because it metabolizes a number of organic compounds, including those found in polluted water. Coincidentally the incidence of PBC is higher in westernized countries (Parikh-Patel, A., et al., Clin Immunol, 91:206-18 (1999)) and may be increasing (Metcalf, J., et al., Semin Liver Dis, 17:13-22 (1997)). Most thories on the environmental contribution to disease have suggested a role for either molecular mimicry via microbial proteins (Gershwin, M E, et al., Immunol Rev, 174:210-25 (2000)) or xenobiotics, with specific halogenated hydrocarbons modifying the lipoic acid residues on PDC-E2 eventually leading to molecular mimicry (Long, S A, et al., J Immunol, 167:2956-63 (2001)).

Molecular mimicry has been defined as the sharing of epitopes on proteins from unrelated species. Such would certainly be the case with the high homology between PDC-E2 from N. aromaticivorans and human PDC-E2. The modification of autoepitopes, from either microrganisms or by xenobiotics in a susceptible individual and in the setting of cryptic T-cells epitopes and degeneracy of T-cell receptors, is a likely mechanism for disruption of tolerance to self antigens in autoimmune conditions (Van de Water, J., et al., Hepatology, 33:771-5 (2001); and Sasaki, M., et al., J Autoimmun, 15:51-60 (2000)). The fact that the liver and the biliary tract are common targets of infections and unique sites for the catabolism of most xenobiotics, as well as a reservoir for bacteria, makes this theory particularly plausible and relevant to PBC.

Viruses and Chlamydia spp. have also been incriminated as possible infectious candidates for initiating PBC (Weber, B., et al., Lancet, 352:149 (1998); and Abdulkarim, A S, et al., (2002)). However, there is no compelling data to suggest such an association (Leung, P S, et al., Hepatology, 36:388A (2002)). In most studies, researchers have focused on cross reactivity of AMA with prokaryotic enzymes involved in the respiratory chain, particularly PDC-E2, because of the significant homology with human PDC-E2. In addition, there has been some epidemiologic evidence suggesting that patients with PBC have a higher incidence of urinary tract infections, although such data remain controversial (Butler, P., et al., Gut, 36:931-4 (1995); and O'Donohue, J., et al., Eur J Clin Microbiol Infect Dis, 16:743-6 (1997)).

The homology described for E. coli PDC-E2 is significantly less than that for N. aromaticivorans (FIG. 1). Further, antibodies to E. coli PDC-E2 appear in the sera of PBC patients later in the disease than antibodies to human PDC-E2, and the titer to E. coli PDC-E2 is significantly less (Fussey, S P, et al., Hepatology, 13:467-74 (1991)). There has also been reactivity shown against non-PDC-E2 bacterial peptides of E. coli (Bogdanos, D P, et al., Liver, 21:225-32 (2001); and Mayo, I., et al., J Hepatol, 33:528-36 (2000)) and L. delbrueckii (Bogdanos, D P, et al., Hepatology, 32:300A (2000)). Such studies have focused only on a limited number of patients or primarily on those with advanced disease. Once again, the titers reported in these other studies are significantly less than against N. aromaticivorans, as described herein.

Xenobiotics are foreign compounds that may either substitute, alter, or complex to self proteins, and thereby induce a structural change to the native structure, which could subsequently break tolerance. Such a response would then be perpetuated by the continuous presence of the native peptide. In fact, recent data have suggested that there are some common halogenated hydrocarbons found in the environment that are reactive against sera from patients with PBC (Long, S A, et al, J Immunol, 167:2956-63 (2001)).

N. aromaticivorans not only has a high degree of homology with the critical lipoylated inner domain of human PDC-E2, but also is capable of metabolizing chemical compounds that are similar to the xenobiotics that have already been shown to be reactive against sera from patients with PBC (Long, S A, et al, J Immunol, 167:2956-63 (2001)). Indeed, patient reactivity against this bacterium has the highest degree of sensitivity and specificity thus far observed and appears to be as reliable as immunoblotting to recombinant mitochondrial proteins in the diagnosis of PBC.

Several further issues highlight the importance of the findings reported herein. First, the reactivity against bacterial homologues of PDC-E2 at titers as high as native human PDC-E2 has never been reported previously against any other biologic agent. Second, it is found in nearly all PBC patients regardless of the disease stage. Third, the sera from a relative of a PBC patient was reactive to N. aromaticivorans at a time when she was clinically asymptomatic, provides support to the thesis that this organism is either the etiologic agent or is a closely related agent.

Fourth, the finding that 12% of AMA-negative sera reacted against N. aromaticiorans also argues that the reactivity is not that of simple homology of amino acid sequences between human and bacterial PDC-E2. This suggests clear evidence of exposure and responsiveness even in the absence of obvious reactivity to PDC-E2. Yet this reactivity has not clearly arisen because of the ubiquity of the bacterium, because of the large number of sera from patients with other conditions or from normal individuals that do not react with the bacterium.

Fifth, the recent finding that Novosphingobium is present in sewage treatment plants, and is involved in the degradation of 17-β estradiol (Fujii, K., et al., Appl Environ Microbiol, 68:2057-60 (2002)), increasing the active fraction, provides an important clue as to the potential mechanisms that may be involved in the pathogenesis of PBC, given the predominance of PBC in women (Kaplan, MM, N Engl J Med, 335:1570-80 (1996)).

N. aromaticivorans (and/or other newly identified bacterial species that possess high homology to human PDC-E2 and degrade environmental hydrocarbons) is responsible for initiating the autoimmune response in PBC. With the increasing use of petrochemicals and insecticides, many of these non-pathogenic bacteria which metabolize environmental hazards, will likely increase their potential of creating neo-protein adducts. Because these bacteria induce an immune response that breaks tolerance without signs of clinical infection.

PBC is a mucosal disease. Asymptomatic infection of women with N. aromaticivorans and a chemical modification of the PDC-E2 inner domain in a genetically susceptible host, would lead to a vigorous self-response, including a local T cell response. This orchestrated response would lead to clinical disease, including the unique apoptotic properties of bile duct cells (Matsumura, S., et al., Hepatology, 35:14-22 (2002)). Those are possible mechanisms by which exposure to N. aromaticivorans breaks tolerance to PDC-E2. During xenobiotic metabolism, bacterial PDC-E2 becomes altered, thus providing an ‘altered self mimic’ to the host immune system and leading to breaking of tolerance.

The second scenario involves the production by N. aromaticivorans of xenobiotic metabolites capable of altering the host PDC-E2, likely through the nucleophilic properties of lipoic acid, thus resulting similarly in the production of altered-self mechanism for the loss of tolerance.

PBC is a multifactorial disease in which not only an organism and/or chemical modifications are required, but also genetic contributions which would facilitate the loss of self tolerance and the subsequent development of a sustained inflammatory response.

Although the invention has been described with reference to the presently preferred embodiment, it should be understood that various modifications can be made without departing from the spirit of the invention. Accordingly, the invention is limited only by the following claims.

1. A method of treating an autoimmune condition comprising administering to a subject in need of such treatment an anti-microbial agent.

2. The method of claim 1, wherein the anti-microbial agent is an anti-bacterial agent.

3. The method of claim 1, wherein the anti-microbial agent is an agent against gram-negative bacteria.

4. The method of claim 1, wherein the anti-microbial agent is an agent against a bacterial strain from the Sphingomonas genus.

5. The method of claim 1, wherein the anti-microbial agent is an agent against a bacterium containing a homologue of human PDC-E2 and capable of metabolizing a compound of polycyclic aromatic hydrocarbons.

6. The method of claim 1, wherein the anti-microbial agent is an anti-N. aromaticivorans agent.

7. The method of claim 1, wherein the anti-microbial agent is an antibody which specifically binds to N. aromaticivorans.

8. The method of claim 1, wherein the autoimmune condition is associated with primary biliary cirrhosis.

9. The method of claim 1, wherein the autoimmune condition is associated with expression of a mitochondrial autoantigen.

10. A method for treating an autoimmune condition comprising administering to a subject in need of such treatment an agent, wherein the agent specifically binds to a complex comprising MHC and a peptide from a protein having an amino acid sequence selected from the group consisting of SEQ ID NO. 1, SEQ ID NO. 2, SEQ ID NO. 3, and SEQ ID NO. 4.

11. The method of claim 10, wherein the agent is a peptide/MHC specific antibody.

12. An antibody which specifically binds to N. aromaticivorans.

13. An antibody which specifically binds to a protein with an amino acid sequence selected from the group consisting of SEQ ID NO. 1, SEQ ID NO. 2, SEQ ID NO. 3, and SEQ ID NO. 4, wherein the antibody does not specifically bind to an epitope of human PDC-E2.

14. An antibody which specifically binds to a complex comprising an MHC molecule and a peptide from a protein having an amino acid sequence selected from the group consisting of SEQ ID NO. 1, SEQ ID NO. 2, SEQ ID NO. 3, and SEQ ID NO. 4, wherein the antibody does not specifically bind to an epitope of human PDC-E2.

15. A method for diagnosing primary biliary cirrhosis in a subject comprising determining the presence of N. aromaticivorans in a sample from a subject, wherein the presence of N. aromaticivorans in the sample is indicative of primary biliary cirrhosis in the subject.

16. The method of claim 15, wherein the presence of N. aromaticivorans in the sample is indicative of early stage primary biliary cirrhosis in the subject.

17. The method of claim 15, wherein the subject is free from a symptom of primary biliary cirrhosis.

18. The method of claim 15, wherein the subject is negative for a test for anti-mitochondrial antibody.

19. The method of claim 15, wherein the subject is negative for a test for mitochondrial auto-antigen.

20. The method of claim 15, wherein the sample is a blood sample.

21. The method of claim 15, wherein determining the presence of N. aromaticivorans in the sample comprises determining the presence of a nucleotide sequence of N. aromaticivorans in the sample.

22. The method of claim 21, wherein the presence of a nucleotide sequence of N. aromaticivorans in the sample is determined by polymerase chain reaction.

23. The method of claim 15, wherein determining the presence of N. aromaticivorans in the sample comprises determining the presence of an amino acid sequence of N. aromaticivorans in the sample.

24. The method of claim 15, wherein determining the presence of N. aromaticivorans in the sample comprises determining the presence of an antibody against N. aromaticivorans in the sample.

25. The method of claim 15, wherein determining the presence of N. aromaticivorans in the sample comprises determining the presence of an activity of N. aromaticivorans in the sample.

26. The method of claim 15 further comprising determining the genetic susceptibility of the subject to primary biliary cirrhosis.

27. The method of claim 15 further comprising determining the subject's exposure to a chemical contaminant.

28. A kit useful for diagnosis or prognosis of primary biliary cirrhosis comprising an agent and an instruction, wherein the agent specifically detects the presence of N. aromaticivorans in a sample.

29. The kit of claim 28, wherein the agent is an antibody which specifically binds to N. aromaticivorans.

30. The kit of claim 28, wherein the agent is linked to a detectable moiety.

31. The kit of claim 28, wherein the agent is an oligonucleotide.

32. The kit of claim 28, wherein the agent is a pair of oligonucleotides useful for a polymerase chain reaction specific to a nucleotide sequence of N. aromaticivorans.

33. The kit of claim 28, wherein the agent is an oligonucleotide linked to a detectable moiety.

34. A method for screening for an agent useful for treating an autoimmune condition comprising screening for an agent capable of binding to N. aromaticivorans or inhibiting the growth of N. aromaticivorans.


SEQUENCE LISTING (22 sequences)

SEQ ID NO: 1 - protein, 489 aa, Novosphingobium aromaticivorans
MRCDMAICPSRSTGIGKFVCGASGNPLDPTRHAGEMAPRKDAGEVSATAPSSLRYQDTENTPMPIAIKMPALSPTMEEGTLAKWLVKVGDKVSSGDIMAEIETDKATMEFEAVDEGTIVSIDVAEGSEGVKVGTVIATLAGEDEDASAPAPKAVAPAAAPVPVPAPKAEPAPAAVSTPAPAAASASKGDRVIATPLAKRIAADKGIDLKGVAGSGPNGRIIRADVEGAKPAAAAPVSTVAPAVASAAAPARAPAAVPDFGIPYEAQKLNNVRKTIARRLTEAKQTIPHIYLTVDIRLDALLKLRGDLNKALEAQGVKLSVNDLIIKALAKALMQVPKCNVSFAGDELRSFKRADISVAVAAPSGLITPIIVDAGSKSVSAIATEMKALANKAREGKLQPHEYQGGTASLSNLGMFSIKNFDAVINPPQAMIMAVGAGEQRPYVIDGALGIATVMSATGSFDHRAIDGADGAELMQAFKNLIENPLGLVA

SEQ ID NO: 2 - protein, 461 aa, Novosphingobium aromaticivorans
MAIELKMPALSPTMEEGTLAKWLVKAGDEVRSGDILAEIETDKATMEFEAVDEGVIAEILVAEGTEGVKVGTVIATIQGEGEDAAPAAATPAVEQKVEMSEAAPSVEARAAPAVAIAPKVDAKPAVDPEIPAGTAMVPTTVREALRDAMAEEMRADDRVFVMGEEVAEYQGAYKVTQGLLDEFGPRRVIDTPITEYGFVGIGAGAAMGGLRPIIEFMTFNFAMQAIDHIINSAAKTNYMSGGQMRCPIVFRGPNGAASRVGAQHSQNYGPWYANVPGLVVIAPYDSADAKGLMKAAIRSEDPVVFLENELVYGRTFDVPQMDDFVLPIGKARIVRQGKDVTIVSYSIGVGLALEAAETLAAEGIDAEVIDLRTLRPLDKDTVLASLAKTNRLVVAEEGFPVCSIASEIMAICMEDGFDHLDAPVLRVCDEDVPLPYAANLEKAALIDAGKIAAAVRKVCYR

SEQ ID NO: 3 - protein, 406 aa, Novosphingobium aromaticivorans
VLNELRIPRMGSVENARLLNWRVQEGEAYEPGQVLYEIETDKTSVEVEAEGPGVLARHLAAVGDEFKVGDRIGLWALPGTAPATLRAALSPQPMPASEPAPSPSSTLPAAVSAPGLHALRPVSRDAAGGRRVSPLARRLAAQNGVDLATVTGTGMGGKISGKDVLAASAKPRPAPVPVSPPRPGSDGEIVPHSLRRRTIAQRMVEAAAIPTLTADMEVDLTALFARRRSVEGNGASVLGMIAEAAIAALLQHRRLNAHWREDAMVQFGAVHLGIAVDTPEGLVVPVVRNAESLNARGLTDAIAALADKARAGTLRPQDMEGGTFTISNPGSMGPVVRAEALLNPPQVALLGLPGIVRAPVAIKDGDAWAMAVRPLLRLSLSFDHRALDGGPVIAFLNTLKATLERP

SEQ ID NO: 4 - protein, 446 aa, Novosphingobium aromaticivorans
MGTYTFRLPDIGEGIAEAEIVAWHVKVGDTVEEDGRLADMMTDKATVEMESPVAGKVVSVAGEVGDVVAIGSALVVIETEGEDEAPAPAAAPAPKAAIVEERIEVETPEPPQPPSPPQPLFVSREVEAPPAVPATGSGVAPGPRASTAPDTIGGAGAKVLASPAVRQRARDLGIDLSEVRPSEEGRIRHADLDQFLSYNASGGYRAAGAERGDEVIRVIGMRRRIAENMAASKRHIPHFSYVEECDVTALEIMREQLNAGRGDKPKLTMLPLLITAICRALPQYPMINARYDDEAGVVTRYGAVHLGMAAQTPAGLMVPVIRNAQTLNLWQLAREIVRLAEAARSGSAKSDELSGSTLTVTSLGPLGGVATTPVINRPEVAIIGPNRIVERPMFVSDGMGGERIEKRKLMNISISCDHRVVDGHDAASFIQAVKKLIETPVLLLAD

SEQ ID NO: 5 - DNA, 28 bp, Artificial sequence
  Amplification primer
aataggatccatgcgctgcgacatggcg

SEQ ID NO: 6 - DNA, 29 bp, Artificial sequence
  Amplification primer
aataaagcttcaggcgaccaggccgagcg

SEQ ID NO: 7 - DNA, 28 bp, Artificial sequence
  Amplification primer
aataggatccatggcaatcgaactgaag

SEQ ID NO: 8 - DNA, 28 bp, Artificial sequence
  Amplification primer
aaaaaagcttcagcgatagcagaccttg

SEQ ID NO: 9 - DNA, 30 bp, Artificial sequence
  Amplification primer
aaaggatccgtgctgaacgaactgcgcatc

SEQ ID NO: 10 - DNA, 29 bp, Artificial
  Amplification primer
aaagaattctcacggccgttccagcgttg

SEQ ID NO: 11 - DNA, 29 bp, Artificial sequence
  Amplification primer
aaatggatccatgggaacctacacattcc

SEQ ID NO: 12 - DNA, 31 bp, Artificial sequence
  Amplification primer
aaatgaattctcagtccgccagcagcagcac

SEQ ID NO: 13 - protein, 14 aa, Homo sapiens
GDLLAEIETDKATI

SEQ ID NO: 14 - protein, 14 aa, Homo sapiens
GDIMAEIETDKATM

SEQ ID NO: 15 - protein, 19 aa, Homo sapiens
LAEIETDKATIGFEVQEEG

SEQ ID NO: 16 - protein, 19 aa, Homo sapiens
LAEIETDKATMEFEAVDEG

SEQ ID NO: 17 - protein, 19 aa, Homo sapiens
LWKKYGDKRIIDTPISEMG

SEQ ID NO: 18 - protein, 14 aa, Homo sapiens
DEVVCEIETDKTSV

SEQ ID NO: 19 - protein, 14 aa, Homo sapiens
GQVLYEIETDKTSV

SEQ ID NO: 20 - protein, 16 aa, Homo sapiens
VQSDKASVTITSRYDG

SEQ ID NO: 21 - protein, 16 aa, Homo sapiens
MMTDKATVEMESPVAG

SEQ ID NO: 22 - protein, 30 aa, Homo sapiens
KVGEKLSEGDLLAEIETDKATIGFEVQEEG